(19) United States
(12) Patent Application Publication
(10) Pub. No.: US 2022/0019313 A1
(43) Pub. Date: Jan. 20, 2022

(54) UNDER-DISPLAY ULTRASOUND BLOOD DYNAMIC PERFORMANCE SENSING

(71) Applicant: SHENZHEN GOODIX TECHNOLOGY CO., LTD., Shenzhen (CN)

(72) Inventors: Yi He, San Diego, CA (US); Bo Pi, San Diego, CA (US)

(21) Appl. No.: 16/931,446

(22) Filed: Jul. 17, 2020

(51) Int. Cl. G06F 3/043; A61B 8/06; G01F 1/66

(52) U.S. Cl. CPC G06F 3/0436; G01F 1/662; A61B 8/06

(57) ABSTRACT

Blood dynamic performance (BDP) sensing is provided using under-display ultrasonic sensors integrated within display panel arrangements of portable electronic devices. For example, a BDP sensing system is integrated in a device to include at least a sensor array of ultrasonic transducers disposed below the display screen and a BDP sensor control circuit. The control circuit can direct the sensor array to transmit ultrasonic waves through a sensing region of the display screen at an object (e.g., a finger) and receive a reflected-back portion of the ultrasonic waves, and generate ultrasonic signals corresponding to the received portion of the ultrasonic waves. A processor can generate BDP output information based on the ultrasonic signals to indicate dynamic performance of blood flowing through at least one of the structures of the object (e.g., heart rate measurements, blood pressure measurements, etc.)

### TECHNICAL FIELD

This disclosure relates to blood performance sensors, and, more particularly, to sensing of blood dynamic performance using under-display sensors, such as ultrasound sensor arrays integrated within display panel arrangements of mobile devices, wearable devices, and other computing devices.

### BACKGROUND

Various sensors can be implemented in electronic devices or systems to provide certain desired functions. Some sensors detect static types of user information, such as fingerprints, iris patterns, etc. Other sensors detect dynamic types of user information, such as body temperature, pulse, etc. The various types of sensors can be used for many different purposes. In some cases, such sensors help enable user authentication, for example, to protect personal data and/or prevent unauthorized access to user devices. In other cases, such sensors can help monitor changes in physical and/or mental state of a user, such as for fitness tracking, biofeedback, etc. To support these and other purposes, various types of sensors can be in communication with, or even integrated with, devices and systems, such as portable or mobile computing devices (e.g., laptops, tablets, smartphones), gaming systems, data storage systems, information management systems, large-scale computer-controlled systems, and/or other computational environments.

As one set of examples, authentication on an electronic device or system can be carried out through one or multiple forms of biometric identifiers, which can be used alone or in addition to conventional password authentication methods. A popular form of biometric identifiers is a person's fingerprint pattern. A fingerprint sensor can be built into the electronic device to read a user's fingerprint pattern so that the device can only be unlocked by an authorized user of the device through authentication of the authorized user's fingerprint pattern. Another example of sensors for electronic devices or systems is a biomedical sensor that detects a biological property of a user, e.g., a property of a user's blood, the heartbeat, in wearable devices like wrist band devices or watches. In general, different sensors can be provided in electronic devices to achieve different sensing operations and functions. Such sensing operations and functions can be used as stand-alone authentication methods and/or in combination with one or more other authentication methods, such as a password authentication, or the like.

Different types of sensors have been integrated in different ways, and to different extents, with mobile electronic devices. For example, many modern smart phones have integrated accelerometers, cameras, and even fingerprint sensors. However, each such sensor integration has involved careful consideration of and compliance with technical, design, and other constraints, such as imposed limits on physical space, power, heat generation, cost, external access (e.g., for sensors relying on physical contact or optical access), interference with interface elements (e.g., a display screen, buttons, etc.), etc.

### SUMMARY

Embodiments provide sensing of blood dynamic performance (BDP) using under-display sensors, such as ultrasound sensor arrays integrated within display panel arrangements of mobile devices, wearable devices, and other computing devices. For example, a portable electronic device (e.g., a smart phone) can include a display screen having functional display layers and a top cover layer configured as a touch screen user interface. A BDP sensing system can be integrated in the device to include at least a sensor array of ultrasonic transducers disposed below the display screen and a BDP sensor control circuit. The control circuit can direct the sensor array to transmit ultrasonic waves through a sensing region of the display screen at an object (e.g., a finger) placed on the sensing region, direct the sensor array to receive a portion of the ultrasonic waves reflected by structures of the object back to the sensor array through the sensing region of the display screen; and generate ultrasonic signals corresponding to the received portion of the ultrasonic waves. A processor coupled with the BDP sensing system can generate BDP output information based on the ultrasonic signals to indicate dynamic performance of blood flowing through at least one of the structures of the object (e.g., heart rate measurements, blood pressure measurements, etc.).

### BRIEF DESCRIPTION OF THE DRAWINGS

The accompanying drawings, referred to herein and constituting a part hereof, illustrate embodiments of the disclosure. The drawings together with the description serve to explain the principles of the invention.

FIG. 1 shows a block diagram of a portable electronic device with under-display blood dynamic performance (BDP) sensing as context for various embodiments described herein.

FIGS. 2A and 2B show an example of a portable electronic device having a BDP sensing system integrated as an under-display BDP sensor, according to various embodiments.

FIG. 3 shows a partial side view of an example configuration of an under-display BDP sensor in context of a finger, according to various embodiments.

FIG. 4 shows top, side, and bottom views of an example of a portable electronic device having multiple BDP sensing systems, according to various embodiments.

FIG. 5 illustrates use of pulse transit time measurements for blood pressure monitoring, according to various embodiments.

FIG. 6 shows a flow diagram of an illustrative method for under-display BDP sensing, according to various embodiments.

In the appended figures, similar components and/or features can have the same reference label. Further, various components of the same type can be distinguished by following the reference label by a second label that distinguishes among the similar components. If only the first reference label is used in the specification, the description is applicable to any one of the similar components having the same first reference label irrespective of the second reference label.

### DETAILED DESCRIPTION

In the following description, numerous specific details are provided for a thorough understanding of the present invention. However, it should be appreciated by those of skill in the art that the present invention may be realized without one or more of these details. In other examples, features and techniques known in the art will not be described for purposes of brevity.

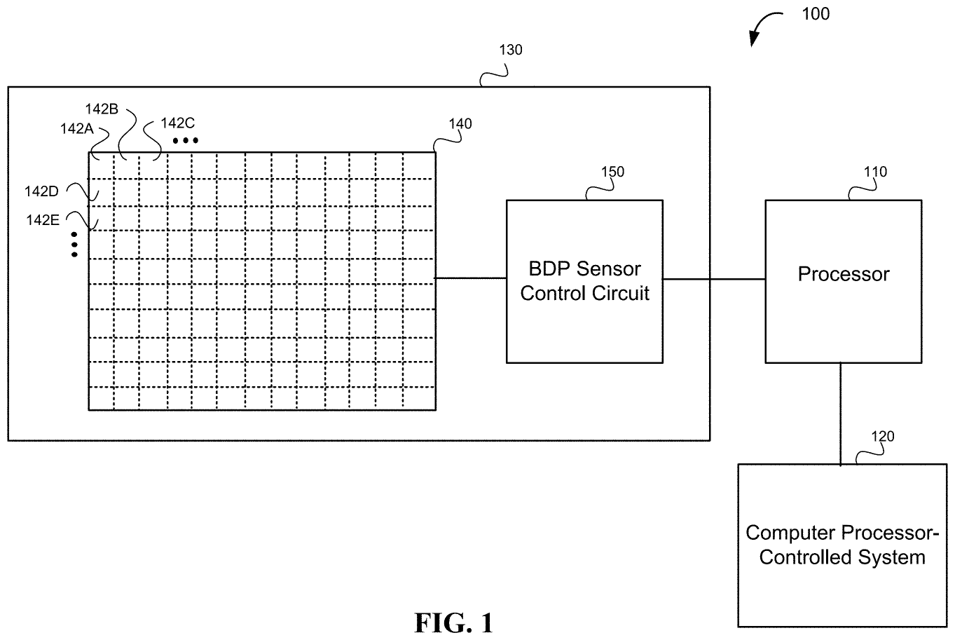

Turning to FIG. 1, a block diagram is shown of a portable electronic device 100 as context for various embodiments described herein. The portable electronic device 100 is illustrated as including a computer processor-controlled system 120 and a blood dynamic performance (BDP) sensing system 130. The computer processor-controlled system 120 is intended generally to represent any suitable system or systems to provide any suitable features of the portable electronic device 100, other than those of the BDP sensing system 130. For example, in a smart phone, the computer processor-controlled system 120 can include subsystems for providing telephonic and communications features, display features, user interaction features, application processing features, etc. Embodiments of the portable electronic device 100 can include one or more processors 110. In some embodiments, the one or more processors 110 are shared between the computer processor-controlled system 120 and the BDP sensing system 130. In other embodiments, one or more processors 110 is used by the computer processor-controlled system 120, and the BDP sensing system 130 has its own one or more dedicated processors 110.

As described herein, the portable electronic device 100 can be equipped with BDP sensing systems 130 to monitor BDP of users. BDP can include pulse, heart rate, blood pressure, and/or any other suitable dynamic performance property of blood as it flow through the body of an individual. Various BDP measurements can be used to support health and/or fitness tracking, liveness sensing in conjunction with biometric authentication, and/or other features. Such portable electronic devices 100 can include any suitable portable or mobile computing devices having at least one integrated display screen, such as smartphones, tablet computers, laptop computers, wrist-worn devices and other wearable or portable devices. The BDP sensing system 130 can include at least one ultrasonic sensing mechanism implemented as an “under-display” BDP sensor by integrating at least one ultrasound sensor in the display of the portable electronic devices, such as under some or all layers of an integrated display assembly. Some implementations of the under-display BDP sensors seek to minimize or eliminate any additional sensing footprint for the ultrasonic sensing, to minimize additional consumption of physical space and power, to avoid interference with display operations and/or other component operations, to support intuitive user interaction, etc.

The inventors of the present application have previously developed novel techniques for detecting heart rate, and the like, based on optical (not ultrasonic, or other acoustic information). For example, some such techniques using optical sensors integrated into mobile electronic devices are described in U.S. Patent Publication No. 2016/0022160, titled “Optical Heart Rate Sensor.” Various optical fingerprint sensors, which detect heartbeat signals and/or other additional biometric markers are described in U.S. Patent Publication Nos. 2018/0046281 and 2018/0173343, both titled “Multifunction Fingerprint Sensor Having Optical Sensing Capability”; and U.S. Patent Publication No. 2017/0316252, titled “Fingerprint Identification Apparatus and Mobile Terminal.” The inventors of the present application also previously developed novel ultrasound-based fingerprint sensing techniques for use with sensors integrated into mobile electronic devices, such as those described in U.S. Patent Publication No. 2018/0314871, titled “Ultrasound Fingerprint Sensing and Sensor Fabrication.” However, unlike the embodiments described herein, these prior approaches were not able to use ultrasound information to monitor blood dynamic performance.

Embodiments of the under-display BDP sensor can include at least a sensor array 140 and a BDP sensor control circuit 150. The sensor array 140 can be implemented as an array of ultrasound transducers. Each ultrasound transducer, or groups of transducers, can be considered as a detector element 142. The BDP sensor control circuit 150 can direct the detector elements 142 to transmit and receive ultrasonic signals. Each detector element 142 can detect responses to the ultrasonic signaling, such as reflected acoustical signal information. For example, ultrasonic information can be reflected back to the detector elements 142 after reflecting off of a physiological feature, such as an artery in a human finger. By mapping the detector elements 142 to respective physical locations in the sensor array 140, detected ultrasonic responses can be used effectively to generate pixels (or groups of pixels) of BDP information. The pixels of BDP information can be passed by the BDP sensor control circuit 150 to the processor(s) 110.

In some embodiments, some or all of the sensor array 140 includes acoustic transducers structured to function both as the acoustic wave source (acoustic transmitters) and as the returned acoustic signal receiver (acoustic receivers). In other embodiments, some or all of the sensor array 140 includes acoustic wave transmitters and returned acoustic signal wave receivers that are separate ultrasound transducers. In some implementations, the ultrasound transducers are arranged in a sensing array built on an integrated circuit (IC) chip, such as a complimentary metal-oxide semiconductor (CMOS) structured chip. For example, the electrodes for each transducer element are prepared on the chip. A single piece, or several large pieces, of ultrasonic transducer materials (e.g., a piezoelectric material) are bounded or coated onto the IC chip. Corresponding electrodes can be connected. The transducer materials are diced or etched to render the discrete ultrasonic transducer elements. Such a design can be configured to realize proper resonant frequency. Gaps among discrete ultrasonic transducer elements can be filled with an appropriate filler material, such as a proper epoxy. The top electrodes of the discrete ultrasonic transducers can then be formed. According to a driving mode, each top electrode can include a single, or several, or a row, or a column of discrete ultrasonic transducer elements. When high voltage is applied to the transducers, ultrasonic waves are generated. For example, a low voltage circuit is connected to the transducers to receive the returned ultrasonic wave induced electric signals. For some implementations using separate transmitting and sensing transducers, separate ultrasound transducer layer structures can be fabricated (e.g., for generating the ultrasound signals and for sensing the ultrasound signals, respectively). For example, in some implementations, a top layer structure is an acoustic signal receiver having ultrasound sensing transducers to detect returned ultrasound signals, and a separate bottom layer structure is an acoustic signal generator having ultrasound emitter transducers to generate the ultrasound signals towards the top sensing area. Some implementations (e.g., in which transducers are configured both to generate and to sense ultrasound signals) further include on-board circuitry (e.g., as part of the BDP sensor control circuit 150) to controlling the transmission and reception functions, such as including a multiplexed driver and receiver architecture.

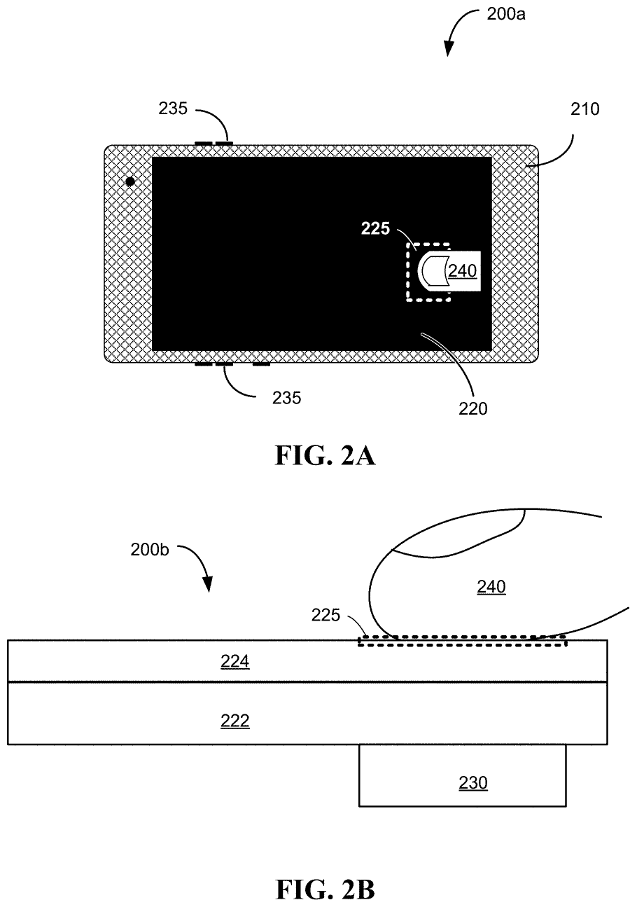

For the sake of illustration, FIGS. 2A and 2B show an example of a portable electronic device 200 having a BDP sensing system 130 integrated as an under-display BDP sensor 230, according to various embodiments. The portable electronic device 200 can be an embodiment of the portable electronic device 100 of FIG. 1, implemented as a smart phone. As illustrated by the top view of the portable electronic device 200 (designated by reference designator 200a in FIG. 2A), embodiments of the portable electronic device 200 include a housing 210 that integrates various features, such as a display screen 220 and one or more physical buttons 235. Any other suitable interface elements can be included in the portable electronic device 200 and integrated with (or within) the housing 210.

As illustrated by the side view of the portable electronic device 200 (designated by reference designator 200b in FIG. 2B), the portable electronic device 200 includes a BDP sensing system 130 disposed under the display screen 220 as an under-display BDP sensor 230. The display screen 220 can include multiple layers, including multiple functional display layers 222 and a top cover layer 224. The multiple functional display layers 222 can implement any suitable type of display, such as a liquid crystal display (LCD), an organic light-emitting diode (OLED) display, a quantum light-emitting diode (QLED) display, a touch-sensitive display (e.g., to implement a capacitive touch-screen), etc. The top cover layer 224 can be any suitable transparent and/or protective layer disposed over the multiple functional display layers 222. In some embodiments, the top cover layer 224 is configured to provide certain features, such as transmission and/or conduction characteristics to support optical features, capacitive features, pressure-sensing features, and/or other features of the display screen 220. Embodiments of the under-display BDP sensor 230 can be installed under some or all of the multiple functional display layers 222.

In some embodiments, the under-display BDP sensor 230, the multiple functional display layers 222, and the top cover layer 224 of the display screen 220 are assembled without air gaps. Many conventional display screen assemblies include air gaps and/or soft components that tend to frustrate transmission of acoustical information (e.g., effectively generate acoustical resistance). For example, air bubbles between the under-display BDP sensor 230 and other layers of the display screen 220 can interfere with ultrasonic detection, for example, by causing scattering of acoustical waves being transmitted and/or received by the under-display BDP sensor 230.

As illustrated in FIGS. 2A and 2B, the location of the sensor array 140 and the manner of integration of the BDP sensing system 130 can define an effective sensing region 225. In some implementations, there is a single under-display BDP sensor 230 with a single sensor array 140 (not explicitly shown) configured to define a single, contiguous sensing region 225. In some implementations, the sensing region 225 is visibly identifiable by a user. For example, the sensing region 225, and/or a boundary around the sensing region 225, is illuminated as a visibly identifiable zone or area for a user to place a finger 240 for ultrasound sensing performed by the under-display BDP sensor 230.

Embodiments of the under-display BDP sensor 230 include one or more sensor arrays 140, such as described with reference to FIG. 1, including one or more arrays of ultrasound transducer elements for generating and/or detecting ultrasonic acoustical information. Embodiments of the under-display BDP sensor 230 also include one or more BDP sensor control circuits 150, such as described with reference to FIG. 1, to control generation and transmission of ultrasound signals by the ultrasound elements of the sensor array 140 and to process return acoustic signals received by the sensor array 140. In some implementations, the BDP sensor control circuit 150 can fully process the received ultrasound signals to generate BDP data. In such implementations, the BDP sensor control circuit 150 can amplify, filter, quantize, convert (e.g., from analog to digital, from one frequency to another, etc.), and/or otherwise pre-process the ultrasound signals. Then, the BDP sensor control circuit 150 can convert the pre-processed signals into BDP measurement data, such as a series of pulse measurement data over time. In some such implementations, the BDP sensor control circuit 150 can then further process the BDP measurement data to generate BDP output data, such as a pulse in beats per minute, or a score (e.g., by applying the BDP measurement data and/or other BDP output data to predefined templates, thresholds, etc.). For example, the BDP output data can indicate (e.g., via the display screen, as information stored to an internal storage, as information transmitted to a remote system, etc.) whether the user's pulse is within a healthy range, whether the user's finger 240 is “live” (e.g., as opposed to a synthetic spoof), etc. In other implementations, the BDP sensor control circuit 150 performs partial processing of the received signals, and communicates the partially processed data to one or more other components or systems for further processing (or storage for later processing). For example, the BDP sensor control circuit 150 can pre-process the ultrasound signals and transmit the pre-processed signals to the one or more processors 110, as described in FIG. 1. The processor(s) 110 can perform further processing, such as generation of one or more types of BDP measurement data, BDP output data, etc. Embodiments of the under-display BDP sensor 230, including implementations of the sensor array 140 and the BDP sensor control circuit 150, can utilize digital data processing functionality, and/or other functionality of the any suitable components of the portable electronic device 200. For example, as described herein, embodiments utilize physical and/or logical ports, communication features, display features, user interface features, etc.

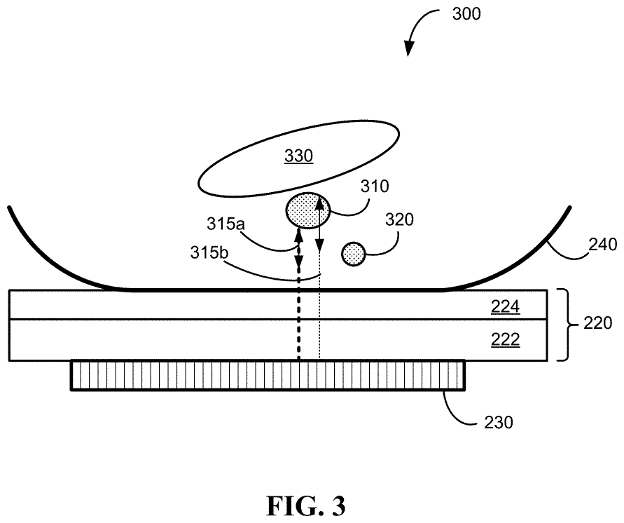

FIG. 3 shows a partial side view 300 of an example configuration of an under-display BDP sensor 230 in context of a finger 240, according to various embodiments. As illustrated, the under-display BDP sensor 230 is disposed below layers of a display screen 220, including multiple functional display layers 222 and a top cover layer 224 (e.g., configured as a touch screen user interface). The under-display BDP sensor 230 can transmit and receive optical signals, such as ultrasonic waves. As described herein, the under-display BDP sensor 230 includes a sensor array of ultrasonic transducers disposed below the display screen 220, and a BDP sensor control circuit (not explicitly shown). The BDP sensor control circuit can be configured to: direct the sensor array to transmit ultrasonic waves through a sensing region of the display screen 220 at the finger 240 (or other object, such as another organ) placed on the sensing region; direct the sensor array to receive a portion of (e.g., some or all of) the ultrasonic waves reflected by structures of the object back to the sensor array through the sensing region of the display screen 220; and generate ultrasonic signals corresponding to the received portion of the ultrasonic waves. A processor (not explicitly shown) can generate BDP output information based on the ultrasonic signals, such as information indicating dynamic performance of blood flowing through at least one of the structures of the object.

In some embodiments, the set of functional display layers 222 and the top cover layer 224 are configured to form an ultrasonic region free of air gaps (e.g., to the extent practical) between the ultrasonic transducers of the sensor array and the top cover layer 224. The ultrasonic region can correspond at least to the sensing region. For example, the illustrated partial side view 300 can represent an ultrasonic region of the configuration in which there are substantially no air gaps.

A human organ is shown in contact with the top layer 224, illustrated as a finger 240. The finger 240 is illustrated as including various anatomical structures, such as an artery 310, a vein 320, and a bone 330. Over a time window with a live finger 240 held in a substantially stable position, it can be predicted that pulsing blood will cause a relatively large and detectable change in the shape of the artery 310, a smaller change in the shape of the vein 320, and no change in the shape of the bone 330.

In operation, the under-display BDP sensor 230 transmits ultrasonic waves through the display screen in the direction of the finger 240. The ultrasonic waves partially transmit into the tissues of the finger 240, interacting with various structures in their paths. For example, frequencies of ultrasonic waves can be tuned to pass through the finger 240 tissue, but to reflect off of structures, such as the artery 310, vein 320, and bone 330. For the sake of illustration, one ultrasonic wave 315a is shown partially reflecting off of a front face of the artery 310, and another ultrasonic wave 315b is shown partially reflecting off of a rear face of the artery 310. The reflected ultrasonic waves can pass back through the display screen in the direction of the under-display BDP sensor 230, and the under-display BDP sensor 230 can detect those reflected ultrasonic waves to generate signals.

The signals generated by the under-display BDP sensor 230 in response to reflected ultrasonic waves can be spatially and/or temporally mapped to generate BDP information over time. In some embodiments, the BDP information is generated by detecting locations where there is a periodic difference in time of transit (ToT) of the ultrasonic signals as an indicator of blood pulse. For example, a ToT is associated with ultrasonic wave 315a reaching the front face of the artery 310 and reflecting back to the under-display BDP sensor 230 for sensing, and a second ToT is associated with ultrasonic wave 315b reaching the rear face of the artery 310 and reflecting back to the under-display BDP sensor 230 for sensing. The difference between the first and second ToT can correspond substantially to the artery 310 diameter. As the artery 310 diameter changes over time due to pulsed blood flow, the ToT difference will change in a corresponding and detectable manner. Typically, the amount of change in ToT difference detected over time in an artery 310 will tend to be appreciably larger than any difference detected in other locations or structures (e.g., in a vein 320 or bone 330). As such, measurements of these changes over time can indicate BDP information, such as pulse. For example, peak detection can be applied to the signals to find periodic peaks in the ToT difference, and a moving average can be computed for the frequency of occurrence of the periodic peaks to determine a heart rate measurement.

In other embodiments, additional or alternative image processing is performed on the signals generated by the under-display BDP sensor 230 (responsive to reflected ultrasonic waves). In some such embodiments, image processing can effectively map the structures of the object (e.g., finger 240) interacting acoustically with the under-display BDP sensor 230. In the case of a live human finger 240, for example, spatial mapping of the acoustical information can be used to form an image of the inner structure of the finger 240, including the artery 310, vein 320, bone 330, etc. Some such embodiments can further temporally map the data to generate a series of time-varying images of the finger 320, from which BDP and/or other information can be obtained. In some embodiments, multiple frequencies are used to penetrate and/or reflect off of different structures and/or materials, and/or to support other features.

In some embodiments, the acoustical information detected and generated by the under-display BDP sensor 230 is used to measure Doppler information. Changes in the blood flow together with changes in arterial diameter (e.g., due to artery 310 inflation) can generate fluctuating Doppler signals. Artery 310 inflation and pulse wave velocity (PWV) in the artery 310 tend to be strongly related to blood pressure. As such, Doppler signals can indicate not only blood pulse performance, but also local blood pressure performance. Typically, blood flow in the veins 320 tends to generate stable Doppler frequency shifts and is not a good indicator of blood pressure performance.

Returning to FIG. 1, some implementations of the BDP sensing system 130 include one or more additional BDP sensors integrated into another portion of the portable electronic devices and/or provided external to (and coupled with) the portable electronic devices. In some embodiments, each additional BDP sensor is implemented as another instance of the BDP sensing system 130, having its own sensor array 140 and BDP sensor control circuit 150. In other embodiments, some or all of the additional BDP sensors can share components, such as sharing one or more BDP sensor control circuits 150. For example, multiple BDP sensors can be implemented essentially as a single sensor array 140 and BDP sensor control circuit 150, where the sensor array 140 is split into multiple, physically separated sub-arrays. In such implementations, the under-display BDP sensor (under-display BDP sensor 230 shown in FIGS. 2A and 2B) and the one or more additional BDP sensors can operate in conjunction to provide additional features, such as monitoring and/or measuring pulse transit time between calibrated organs so as to monitor blood pressure. Embodiments of the one or more additional BDP sensors can be substantially identical to the under-display BDP sensor 230. For example, each of the one or more additional BDP sensors can include substantially the same sensing and/or processing components as the under-display BDP sensor 230, but arranged or implemented as appropriate for its implementation context (e.g., in accordance with its respective position, orientation, structural support, housing, etc.). In other embodiments, some or all of the one or more additional BDP sensors can be appreciably different from the under-display BDP sensor 230.

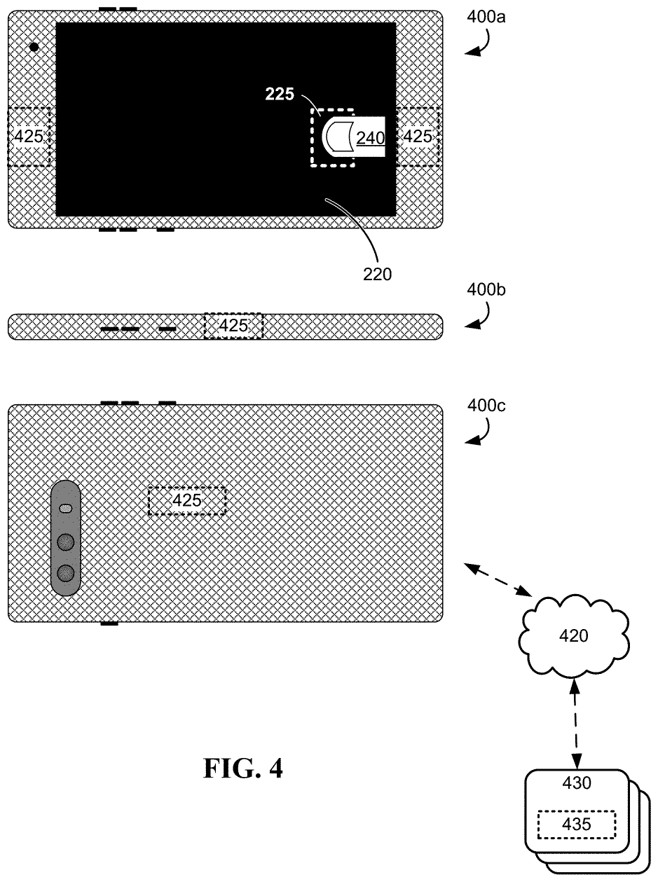

For the sake of illustration, FIG. 4 shows top, side, and bottom views of an example of a portable electronic device 400 having multiple BDP sensing systems, according to various embodiments. The portable electronic device 400 can be an embodiment of the portable electronic device 100 of FIG. 1, implemented as a smart phone. Similar to the portable electronic device 200 of FIGS. 2A and 2B, the portable electronic device 400 includes an under-display BDP sensor 230 (only the corresponding sensing region 225 is explicitly shown) disposed under a display screen 220. The portable electronic device 400 also includes one or more auxiliary-integrated BDP sensors 425. To avoid over-complicating the Figure, each dashed regions corresponding to each instance of reference designator 425 can be considered as an approximate location at which a corresponding auxiliary-integrated BDP sensor 425 can be located, and/or as a corresponding sensing region defined by (e.g., serviced by) that auxiliary-integrated BDP sensor 425.

For example, the top view of the portable electronic device 400a shows one or more auxiliary-integrated BDP sensors 425 as within the housing of the portable electronic device 400 in locations at least partially outside of the edge of the display screen 225. Those one or more auxiliary-integrated BDP sensors 425 can each define a respective auxiliary-integrated sensing region on a same face of the portable electronic device 400 as that of the under-display BDP sensor 230, but not under the display screen 225. Similarly, the side view of the portable electronic device 400b shows an auxiliary-integrated BDP sensor 425 as within the housing of the portable electronic device 400 at a location around the side periphery of the display screen 225. Such an auxiliary-integrated BDP sensor 425 can define a respective auxiliary-integrated sensing region on the side of the portable electronic device 400. Similarly, the bottom view of the portable electronic device 400c shows an auxiliary-integrated BDP sensor 425 as within the housing of the portable electronic device 400 at a location below the display screen 220 (and/or at a location around the periphery of the display screen), but defining a respective auxiliary-integrated sensing region on the back of the portable electronic device 400 (e.g., ultrasonic transmission by the sensor array 140 is in a direction substantially opposite that of the under-display BDP sensor 230).

As described herein, structures of the display screen 220 can frustrate operation of the under-display BDP sensor 230. For example, air gaps and/or soft structures can effectively scatter or otherwise interfere with transmission and/or receipt of ultrasonic signals. Embodiments of the auxiliary-integrated BDP sensors 425 can be positioned and oriented in locations within the housing that are relatively favorable to ultrasonic transmission. For example, ultrasonic signals may be more easily and/or reliably transmitted and sensed by sensor arrays 140 of the one or more of the auxiliary-integrated BDP sensors 425 disposed around the periphery of the display screen 220, or in other locations and/or orientations. In some implementations in which better (e.g., more reliable, more discernable) acoustic information can be obtained from one or more of the auxiliary-integrated BDP sensors 430 than from the under-display BDP sensor 230, acoustical information from the one or more of the auxiliary-integrated BDP sensors 430 can be used to augment, validate, and/or otherwise support processing of the acoustic information sensed by the under-display BDP sensor 230.

In some embodiments, the portable electronic device 400 also or alternatively includes one or more auxiliary-connected BDP sensors 430, each defining one or more respective auxiliary-connected sensing region 435. The auxiliary-connected BDP sensors 430 can be in communication with the portable electronic device 400 via a local network 420. The local network 420 can include any suitable wired and/or wireless links to facilitate communication of BDP information from the auxiliary-connected BDP sensors 430 to the portable electronic device 400 and/or instructions or other information from the portable electronic device 400 to the auxiliary-connected BDP sensors 430. In some implementations, the auxiliary-connected BDP sensors 430 include dedicated ultrasound sensing systems, such as a portable ultrasound device configured to couple (via a physical port and/or wirelessly) with the portable electronic device 400. In other implementations, the auxiliary-connected BDP sensors 430 include multi-function devices that include ultrasound sensing systems. For example, an auxiliary-connected BDP sensor 430 can be implemented as an integrated functionality of a smart phone, tablet computer, wearable device, fitness tracker, etc.

In some implementations, one or more of the auxiliary-integrated BDP sensors 425 and/or auxiliary-connected BDP sensors 430 can use techniques other than ultrasound to obtain BDP information. For example, one or more auxiliary-integrated BDP sensors 425 and/or auxiliary-connected BDP sensors 430 can include optical sensors, capacitive sensors, and/or other sensors. In some implementations, one or more of the auxiliary-integrated BDP sensors 425, auxiliary-connected BDP sensors 430, and/or under-display BDP sensor can obtain information in addition to (or instead of) BDP information. For example, among multiple sensors, data can be obtained relating to one or more fingerprints, body temperature, etc. In one implementation, the under-display BDP sensor obtains both fingerprint information and BDP information.

Embodiments of the BDP sensing system 130 (e.g., including the under-display BDP sensor 230, and, in some implementations, also including one or more auxiliary-integrated BDP sensors 425 and/or auxiliary-connected BDP sensors 430) can include, and/or support interfacing with any hardware and software necessary to obtain BDP data, such as heart rate, pulse, blood pressure, etc. Using multiple sensors, embodiments can combine the under-display BDP sensor 230 data with sensor data from other sensors to enhance the accuracy of data analysis and/or to generate data not detectable with a single sensor, and/or to provide relevant feedback information to a user. For example, the BDP sensing system 130 can be used to detect a user's heart rate, which can be combined with other sensor data, such as motion and other biometric sensors from the same portable electronic device (and/or auxiliary-connected BDP sensors 430), to more accurately track health- or fitness-related activities (e.g., running, swimming, walking, etc.) by measuring both an extent of a user's motion and the intensity of the user's motions during the activity. Though not explicitly shown, some embodiments of the portable electronic devices described herein can be communicatively coupled with one or more remote computational systems via one or more networks. For example, certain data processing features can involve communicating data with, and/or storing data to, a cloud server.

As described herein, the BDP sensing system 130 operates to monitor at least dynamic performance characteristics of blood, such as heartbeat or heart rate (e.g., pulse), living organism detection, pressure force, vessel dynamic performances, local blood flow velocity, etc. Some or all of these BDP characteristics can be measured in multiple locations on a user's body, such as via a fingertip, other finger portions, wrist, neck, chest, etc. For example, when the user's heart beats, the pulse pressure pumps the blood to flow in arteries and veins throughout the body. The changes in blood flow cause detectable anatomic features. For example, arteries tend to change shape as the blood flows through them. This blood flow, and changes in this blood flow, can be detected using at least ultrasonic techniques. As one example, detecting anatomical changes characteristic of blood flow can indicate detection of a living organism. As another example, measuring periodicity (e.g., frequency) of expansion and contraction of arteries over a time window can be used as a proxy for measuring heart rate. As another example, changes in the force applied by a user via the user's finger can affect blood flow through arteries in the finger (e.g., or any other suitable body part), such as by altering a difference between arterial and venous blood flow. Measuring and comparing arterial and venous blood flow signals can thus be used as a proxy for measuring pressure being applied by a user.

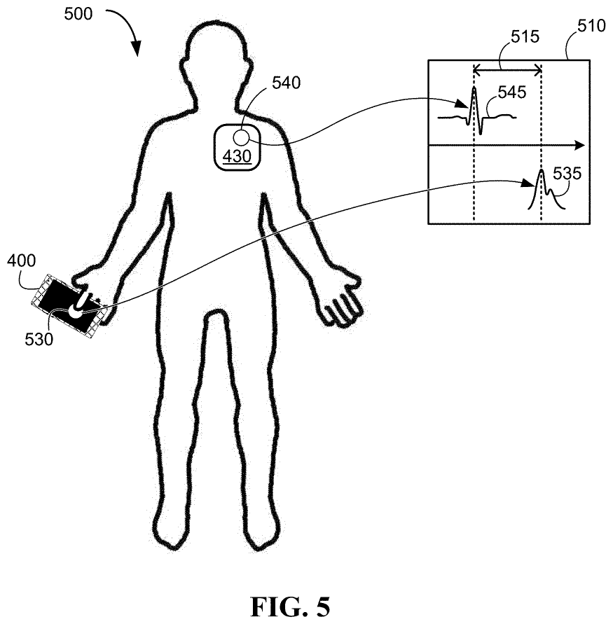

As another example, detected phase delays between a user's heart rate measured in different locations (e.g., according to pulse transit time) can be used as proxy for measuring blood pressure. FIG. 5 illustrates use of pulse transit time measurements for blood pressure monitoring, according to various embodiments. A human body 500 is shown, along with an instance of the portable electronic device 400 (e.g., which can similarly represent portable electronic device 100 or portable electronic device 200) and an instance of an auxiliary-connected BDP sensor 430. The portable electronic device 400 includes at least an under-display BDP sensor 230 (not explicitly shown), onto which the human user has placed a fingertip. As such, BDP data generated by the under-display BDP sensor 230 corresponds to a fingertip measurement location 530. An auxiliary-connected BDP sensor 430 is positioned to measure data substantially at the human user's carotid artery, such that BDP data generated by the auxiliary-connected BDP sensor 430 corresponds to a carotid measurement location 540.

FIG. 5 also shows an example plot 510 of measured BDP information over a time window. A first partial waveform 545 shows an illustrative blood pulse derived from ultrasonic information generated by the auxiliary-connected BDP sensor 430 at the carotid measurement location 540. A second partial waveform 535 shows an illustrative blood pulse derived from ultrasonic information generated by the under-display BDP sensor 230 at the fingertip measurement location 530. It is assumed that the two partial waveforms represent the same blood pulse. A pulse of blood pumped from the heart travels through the arteries of the body at a pulse wave velocity (PWV), and it takes different amounts of time for the pulse to reach different organs and/or parts of the body. The blood pulse transit time (PTT) from the heart to each other organ can be strongly correlated to blood pressure. As illustrated by the plot 510, a particular PTT measurement 515 can be derived by comparing timing differences between the partial waveforms.

While the specific example shows a fingertip measurement location 530 and a carotid measurement location 540, any locations at which BDP information can be reliably measured can be selected. Embodiments can provide for selection and/or calibration of different locations. For example, in connection with measuring BDP information, embodiments can prompt a user to provide location data, such as by selecting from a pre-determined list of candidate and/or pre-calibrated locations. With the sensing technology progress, the PTT between two organs can also be calibrated to measure the blood pressure. As one example, referring to FIG. 4, different portions of a same finger 240 can lie on sensing regions of both the under-display BDP sensor 230 and an auxiliary-integrated BDP sensor 425 adjacent to the display periphery. As another example, one finger can lie on the sensing region of the under-display BDP sensor 230, while another finger lies on an auxiliary-integrated BDP sensor 425 on the rear of the portable electronic device 400. As another example, a finger can lie on the sensing region of the under-display BDP sensor 230, while other BDP information is obtained at the user's wrist by a smart watch or fitness tracker bracelet, or at the user's ear by smart earphones, etc.

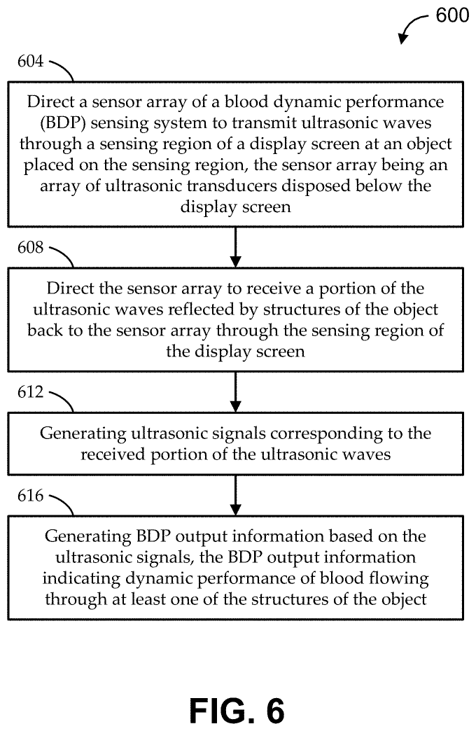

FIG. 6 shows a flow diagram of an illustrative method 600 for under-display blood dynamic performance (BDP) sensing, according to various embodiments. Embodiments of the method 600 begin at stage 604 by directing a sensor array of a BDP sensing system to transmit ultrasonic waves through a sensing region of a display screen at an object placed on the sensing region, the sensor array being an array of ultrasonic transducers disposed below the display screen. At stage 608, embodiments can direct the sensor array to receive a portion of the ultrasonic waves reflected by structures of the object back to the sensor array through the sensing region of the display screen. At stage 612, embodiments can generate ultrasonic signals corresponding to the received portion of the ultrasonic waves.

At stage 616, embodiments can generate BDP output information based on the ultrasonic signals. The BDP output information can indicate dynamic performance of blood flowing through at least one of the structures of the object, such as through an artery of a human finger. In some embodiments, the generating at stage 616 includes obtaining the ultrasonic signals over a time window and mapping the ultrasonic signals to a time basis. In some embodiments, the generating at stage 616 includes spatially mapping of the ultrasonic signals based on a spatial distribution of the ultrasonic transducers to form a dynamically changing image of at least one of the structures of the object. For example, the spatial mapping can effectively produce a changing image of an artery, which can be analyzed using image processing techniques to detect blood pulse, or other BDP information.

In some embodiments, the generating at stage 616 includes analyzing the ultrasonic signals to identify at least a first ultrasound signal corresponding to first respective time of travel (ToT) data over time and a second ultrasound signal corresponding to second respective ToT data over the time; and generating the BDP output information to indicate a heart rate based on determining a pattern of dynamic change in a difference between the first respective ToT data and the second respective ToT data over the time. For example, the relevant ultrasound signals can be identified based at least on determining that the pattern of dynamic change in the difference between the first respective ToT data and the second respective ToT data is characteristic of an arterial location. In some embodiments, the sensor array is a first sensor array of multiple sensor arrays of the BDP sensing system, each defining a respective sensing region of multiple sensing regions. In such embodiments, the generating at stage 616 can include: receiving first ultrasonic signals from the first sensor array; receiving second ultrasonic signals from a second sensor array; temporally comparing the first ultrasonic signals and the second ultrasonic signals to determine a blood pulse transit time (PTT); and generating the BDP output information to indicate a blood pressure based at least on the determined blood PTT.

While this disclosure contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

A recitation of “a”, “an” or “the” is intended to mean “one or more” unless specifically indicated to the contrary. Ranges may be expressed herein as from “about” one specified value, and/or to “about” another specified value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. When such a range is expressed, another embodiment includes from the one specific value and/or to the other specified value. Similarly, when values are expressed as approximations, by use of the antecedent “about,” it will be understood that the specified value forms another embodiment. It will be further understood that the endpoints of each of the ranges are included with the range.

All patents, patent applications, publications, and descriptions mentioned here are incorporated by reference in their entirety for all purposes. None is admitted to be prior art.

What is claimed is:

1. A system for integration in a portable electronic device that includes a display screen, the system comprising:
a blood dynamic performance (BDP) sensing system comprising a sensor array of ultrasonic transducers configured to be located below the display screen in the portable electronic device, and a BDP sensor control circuit configured to:
direct the sensor array to transmit ultrasonic waves through a sensing region of the display screen at an object placed on the sensing region;
direct the sensor array to receive a portion of the ultrasonic waves reflected by structures of the object back to the sensor array through the sensing region of the display screen; and
generate ultrasonic signals corresponding to the received portion of the ultrasonic waves; and
a processor coupled with the BDP sensing system to generate BDP output information based on the ultrasonic signals, the BDP output information indicating dynamic performance of blood flowing through at least one of the structures of the object.

2. The system of claim 1, wherein:
each of the ultrasonic transducers is configured to selectively operate in a first operating mode and a second operating mode;
the BDP sensor control circuit is configured to direct the sensor array to transmit the ultrasonic waves by directing the ultrasonic transducers to operate in the first operating mode; and
the BDP sensor control circuit is configured to direct the sensor array to receive the portion of the ultrasonic waves by directing the ultrasonic transducers to operate in the second operating mode.

3. The system of claim 1, wherein:
a first subset of the ultrasonic transducers is configured to transmit the ultrasonic waves; and
a second subset of the ultrasonic transducers is configured to receive the portion of the ultrasonic waves, the second subset being disjoint from the first subset.

4. The system of claim 1, wherein the processor is to generate the BDP output information at least by obtaining the ultrasonic signals over a time window and mapping the ultrasonic signals to a time basis.

5. The system of claim 1, wherein the processor is to generate the BDP output information at least according to a spatially mapping of the ultrasonic signals based on a spatial distribution of the ultrasonic transducers.

6. The system of claim 1, wherein the processor is to generate the BDP output information at least by:
analyzing the ultrasonic signals to identify at least a first ultrasound signal corresponding to first respective time of travel (ToT) data over time and a second ultrasound signal corresponding to second respective ToT data over the time; and
generating the BDP output information to indicate a heart rate based on determining a pattern of dynamic change in a difference between the first respective ToT data and the second respective ToT data over the time.

7. The system of claim 1, wherein:
the BDP sensing system comprises a plurality of sensor arrays, each defining a respective sensing region of a plurality of sensing regions, the sensor array of ultrasonic transducers disposed below the display screen being a first sensor array, the sensing region of the display screen being a first sensing region; and
the processor is to generate the BDP output information at least by:
receiving first ultrasonic signals from the first sensor array;
receiving second ultrasonic signals from a second sensor array;
temporally comparing the first ultrasonic signals and the second ultrasonic signals to determine a blood pulse transit time (PTT); and
generating the BDP output information to indicate a blood pressure based at least on the determined blood PTT.

8. The system of claim 7, wherein the first sensor array and the second sensor array are integrated in a unitary portable electronic device.

9. The system of claim 7, wherein:
the first sensor array is integrated in a first portable electronic device; and
the second sensor array is integrated in a second portable electronic device that is physically separate from the first portable electronic device and is communicatively coupled with the first portable electronic device via a local communication link.

10. The system of claim 9, wherein the local communication link comprises a wireless communication link of a local wireless network.

11. The system of claim 1, further comprising:
the display screen, including a set of functional display layers and a top cover layer.

12. The system of claim 11, further comprising:
a portable electronic device housing having the display screen, the BDP sensing system, and the processor integrated therein.

13. The system of claim 11, wherein the sensor array, the set of functional display layers, and the top cover layer are assembled without air gaps over at least an ultrasonic region, the ultrasonic region corresponding at least to the sensing region.

14. The system of claim 11, wherein the display screen is a liquid crystal display screen.

15. A method for under-display blood dynamic performance sensing, the method comprising:
directing a sensor array of a blood dynamic performance (BDP) sensing system to transmit ultrasonic waves through a sensing region of a display screen at an object placed on the sensing region, the sensor array being an array of ultrasonic transducers disposed below the display screen;
directing the sensor array to receive a portion of the ultrasonic waves reflected by structures of the object back to the sensor array through the sensing region of the display screen;
generating ultrasonic signals corresponding to the received portion of the ultrasonic waves; and
generating BDP output information based on the ultrasonic signals, the BDP output information indicating dynamic performance of blood flowing through at least one of the structures of the object.

16. The method of claim 15, wherein the generating the BDP output information comprises obtaining the ultrasonic signals over a time window and mapping the ultrasonic signals to a time basis.

17. The method of claim 15, wherein the generating the BDP output information comprises spatially mapping of the ultrasonic signals based on a spatial distribution of the ultrasonic transducers to form a dynamically changing image of at least one of the structures of the object.

18. The method of claim 15, wherein the generating the BDP output information comprises:
analyzing the ultrasonic signals to identify at least a first ultrasound signal corresponding to first respective time of travel (ToT) data over time and a second ultrasound signal corresponding to second respective ToT data over the time; and
generating the BDP output information to indicate a heart rate based on determining a pattern of dynamic change in a difference between the first respective ToT data and the second respective ToT data over the time.

19. The method of claim 18, wherein the identifying is based at least on determining that the pattern of dynamic change in the difference between the first respective ToT data and the second respective ToT data is characteristic of an arterial location.

20. The method of claim 15, wherein:
the sensor array is a first sensor array of a plurality of sensor arrays of the BDP sensing system, each defining a respective sensing region of a plurality of sensing regions; and
the generating the BDP output information comprises:
receiving first ultrasonic signals from the first sensor array;
receiving second ultrasonic signals from a second sensor array;
temporally comparing the first ultrasonic signals and the second ultrasonic signals to determine a blood pulse transit time (PTT); and
generating the BDP output information to indicate a blood pressure based at least on the determined blood PTT.
